(19) United States
(12) Patent Application Publication
(10) Pub. No.: US 2013/0195249 A1
(43) Pub. Date: Aug. 1, 2013

(54) Medical System

(71) Applicants: Wolfgang Edler, Erlangen (DE); Joachim Grottel, Lauf (DE); Harald Karl, Furth (DE); Heiko Mehldau, Nurnberg (DE)

(72) Inventors: Wolfgang Edler, Erlangen (DE); Joachim Grottel, Lauf (DE); Harald Karl, Furth (DE); Heiko Mehldau, Nurnberg (DE)

(21) Appl. No.: 13/750,711

(22) Filed: Jan. 25, 2013

(30) Foreign Application Priority Data
Jan. 27, 2012 (DE) DE 102012201222.2

(51) Int. Cl. A61B 6/00

(52) U.S. Cl. CPC A61B 6/54
USPC 378/91

(57) ABSTRACT

A medical system such as a diagnostic or a therapeutic system includes a fixed base unit with a control unit and a mobile assembly mounted rotatably on the fixed base unit. The fixed base unit and the mobile assembly are set up to exchange data. Transmission of first control data from the control unit to the mobile assembly takes place via a first transmission path. Transmission of second control data to the control unit and transmission of measured data from the mobile assembly takes place via a second transmission path.

This application claims the benefit of DE 10 2012 201 222.2, filed on Jan. 27, 2012, which is hereby incorporated by reference.

### BACKGROUND

The present embodiments relate to a medical system including a fixed base unit with a control unit and a mobile assembly, where the base unit and the assembly are configured to exchange data.

With a computed tomography scanner, image data is transmitted from a gantry that rotates at several revolutions a second during an examination, to a stationary structure or a fixed base unit. Control data is exchanged between the gantry and the base unit.

Three unidirectional transmission paths are used to exchange the data (e.g., one transmission path for the transmission of the image data, one transmission path for the transmission of control data to the gantry, and one transmission path for the transmission of control data to the base unit). Alternatively, the transmission of the image data takes place unidirectionally with a data rate of, for example, >1 Gbit/s, and the transmission of the control data takes place bidirectionally between the stationary structure and the rotating gantry. A data rate between 250 kbit/s and one Gbit/s may be used.

The rotation of the gantry with respect to the stationary structure during an examination provides that simple cabling may not be used for the data exchange to the interface. Instead, the data transmission takes place via sliding contacts (e.g., slip rings with contacts or via contactless data transmission links (CDTs); using a capacitive coupling of a transmitter and receiver in the near field).

A data transmission embodiment of this kind is found not only with computed tomography scanners, but also with other medical systems including a mobile modular unit and a fixed base unit (e.g., with therapeutic devices for oncology, some of which are also used with a rotating gantry).

### SUMMARY AND DESCRIPTION

The present embodiments may obviate one or more of the drawbacks or limitations in the related art. For example, an alternative embodiment of a medical system is provided.

The medical system is, for example, a diagnostic or therapeutic system including a base unit (e.g., a fixed based unit) with a control unit and a mobile assembly (e.g., mounted rotatably on the base unit). The base unit and the assembly exchange data and are set up such that the transmission of first control data from the control unit to the mobile assembly takes place via a first transmission path, and the transmission of second control data to the control unit and the transmission of measured data from the mobile assembly takes place via a second transmission path. Common transmission of the second control data and the measured data via a common transmission path or a common transmission channel enables the number of transmission paths or transmission channels to be reduced, thus resulting in reduced production costs for a corresponding medical system and in increased reliability of the same.

A transmission path or transmission channel may, for example, be a contactless data transmission link with a capacitive coupling or a sliding contact. In the case of the use of sliding contacts from the prior art for the transmission of the first control data toward the mobile assembly and the second control data from the mobile assembly, a separate sliding contact is provided in each case.

With the medical system described, no separate transmission path (e.g., no separate sliding contact) is provided for the second control data.

In one embodiment of the medical system, the measured data corresponds to image data in an imaging system. The volume of image data to be transmitted per time unit (e.g., a data rate) may be more than 1 Gbit/s, while the volume of control data to be transmitted per time unit may be between 250 kbit/s and 1 Gbit/s. The volume of control data to be processed and transmitted may, therefore, be substantially lower than the volume of measured data to be processed. The control data does not contain any image data, but is used to control the controllable functional units of the assemblies. In the case of a computed tomography scanner, for example, the first control data controls the movement of the gantry, and the X-ray source and the second control data contains sensor data relevant for the control of the gantry (e.g., reference values for control loops). As a result, at least in the case of the measured data, this uses a transmission path configured for relatively high data rates. If the transmission of the second control data is combined with the transmission of the measured data, due to the relatively low data rate with the control data, there is hardly any change in the transmission path. The transmission path for the first control data may be implemented more simply since the transmission path is configured for a relatively low data rate. The requirements for a bidirectional transmission path are significantly higher in the case of a combination of the transmission of the first control data with the transmission of the second control data.

In one embodiment of the medical system, the two transmission paths are set up as unidirectional transmission paths. Unidirectional communication connections are considered to be more efficient than bidirectional communication connections and may also be implemented more simply. However, in certain cases, unidirectional transmission paths are also more susceptible to errors during data transmission, since the unidirectional transmission paths are unable to provide any feedback. Although, in the case of the embodiment of the medical system where two unidirectional transmission paths are provided, the transmission of the control data is divided in a directionally oriented manner between two unidirectional transmission paths so that, together, bidirectional data exchange effectively takes place at least for the control data. Therefore, the use of two unidirectional transmission paths virtually represents bidirectional data transmission. This enables the advantages of unidirectional communication to be partially combined with the advantages of bidirectional communication for the control data of the medical system.

In addition, it is advantageous for the transmission of the data via at least one of the transmission paths to take place in a contactless manner (e.g., using capacitive coupling). A contactless transmission path may be less susceptible to defects than an embodiment of a transmission path using a sliding contact and enables larger data volumes to be transmitted significantly more simply. Consequently, the second control data and the measured data may be transmitted via a contactless transmission path (e.g., established by capacitive coupling).

In one embodiment of the medical system, the second control data and the measured data are transmitted with a frequency offset via the second transmission path. The second control data and the measured data are each provided with a frequency band, and the data transmission of the corresponding data may take place permanently. In this way, the second control data is substantially transmitted continuously, thus avoiding any time delay due to a data logjam. This is, for example, advantageous if the second control data includes sensor data that is used to determine a system deviation in a control loop.

According to a further variant of the medical system, the second control data for the transmission via the second transmission path may be upmodulated to a carrier frequency. In this case, the measured data is transmitted unmodulated, thus enabling the technical effort associated with modulation and demodulation to be reduced. In this case, modulation and demodulation, such as is usual with contactless data transmission, is only performed for the second control data. This is, for example, used for the frequency-based separation of the two data streams.

In a further variant of the medical system, the second control data and the measured data are transmitted with a time offset via the second transmission path. The data transmission may take place without an enforced frequency offset between the second control data and the measured data. This enables the frequency bandwidth for the second transmission path to be reduced, thus favoring a simpler technical implementation.

In one embodiment, the second control data and the measured data are each divided into data packets, and the transmission of the data packets is performed in correspondingly provided time windows. One data packet is transmitted in each time window. In this case, two time-window types with different extensions arranged in a periodic sequence (e.g., consecutively in alternation) may be used for the time-offset transmission of the data. A first time-window type is provided for the second control data, and a second time-window type is provided for the measured data. For example, a data packet with second control data and a data packet with measured data are transmitted in alternation.

Each time window for the data packets with control data has a larger temporal extension than the time window for the data packets with measured data in order to take account of the typically different data rates.

Alternatively, time windows of the same extension are provided for the time-offset transmission, and the data packets with second control data are transmitted in a periodic sequence. This provides that, for example, each third time window for data packets is provided with second control data, while the other time windows are reserved for the measured data.

In another variant of the medical system, the second control data and the measured data are each buffered in a buffer memory that accesses a data packet generator in such a way that, in a periodic sequence, either data from the buffer memory for the second control data or data from the buffer memory for the measured data are combined to form one data packet. The one data packet is transmitted in a time window with a given extension. Preference is given to a controllable data packet generator, with which the periodic sequence may be varied in order to be able to take account of actually occurring and possibly varying data volumes in the cases of both the second control data and the measured data.

### BRIEF DESCRIPTION OF THE DRAWINGS

FIG. 1 shows a medical system from the prior art with a fixed base unit and a rotatable assembly;

FIG. 2 shows one embodiment of a medical system with a fixed base unit and a rotatable assembly;

FIG. 3 shows a block diagram of a variant of a second transmission path; and

FIG. 4 shows a block diagram of an alternative variant of the second transmission path.

### DETAILED DESCRIPTION OF THE DRAWINGS

Corresponding parts are each given the same reference numbers in all the figures.

A medical system 2 described below is, for example, a computed tomography scanner with a fixed base unit 4 and an assembly (e.g., a gantry 6) mounted rotatably on the fixed base unit 4.

The fixed base unit 4 includes a control unit 8 that is used to control the medical system 2 and to which an operator may send commands via an operator console (not shown). The fixed base unit 4 also includes an image processing unit 10 that is used for processing image data BD acquired by an image generation unit 12 including an X-ray source and an X-ray detector during each examination.

The image data generation using the image generation unit 12 takes place in the gantry 6, which houses an X-ray detector and an X-ray source. For the generation of image data BD during each examination of a patient, the X-ray source and the X-ray detector are controlled to provide that the image data generation takes place as desired. As a result, data is exchanged between the gantry 6 and the fixed base unit 4. Image data BD is to be transmitted from the gantry 6 on the fixed base unit 4. Control data ESD, ZSD are to be transferred from the fixed base unit 4 toward the gantry 6 and from the gantry 6 toward the fixed base unit 4. In the case of the control data ESD, ZSD, therefore, data exchange takes place in both directions, which, for example, enables control loops for the specification of parameters to be achieved. For example, a reference value is specified by the control unit 8 in the fixed base unit 4, and an actual value is detected by sensors by a control data unit 14 in the gantry 6.

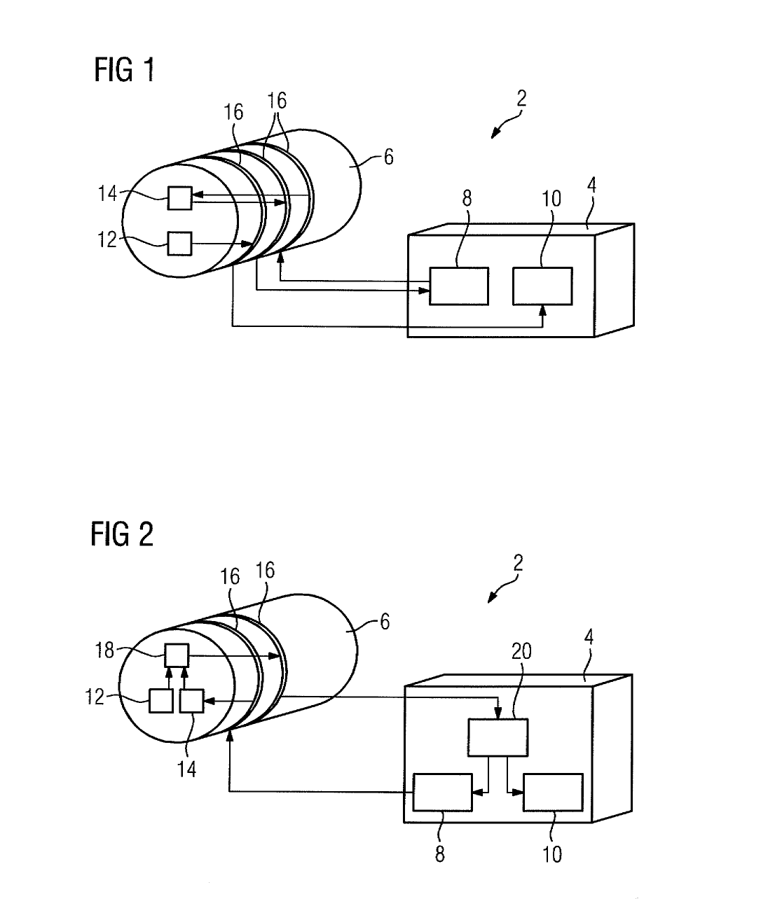

A common solution for data transmission between the fixed base unit 4 and the gantry 6 is shown schematically in FIG. 1. In this case, a separate transmission path or transmission channel is provided in each case for an image data transmission from the gantry 6 toward the base unit 4, for a data transfer of first control data ESD from the base unit 4 toward the gantry 6, and for transmission of second control data ZSD from the gantry 6 toward the base unit 4. Signaling coupling between the gantry 6 and the fixed base unit 4 takes place, as indicated via sliding contacts 16 encircling the gantry 6. Each transmission path has its own sliding contact 16.

By contrast, with a medical system 2 described here, only two transmission paths or transmission channels are provided for the data exchange between the fixed base unit 4 and the gantry 6, thus saving one transmission path or transmission link resulting in a reduction of the technical effort for the implementation of a corresponding medical system 2, reduced production costs and increased reliability in operation.

FIG. 2 shows a corresponding embodiment of a medical system 2 of this kind (e.g., a computed tomograph scanner). According to this illustration, a first transmission path is set up for the transmission of first control data ESD from the control unit 8 of the fixed base unit 4 to the control data unit 14 of the gantry 6, and a second transmission path is provided for the transmission of data from a coupling unit 18 of the gantry 6 toward a decoupling unit 20 of the base unit 4. The coupling unit 18 couples the second control data ZSD from the control data unit 14 of the gantry 6 and the image data BD of the image generation unit 12, and the second control data ZSD and the image data BD are transmitted via a single transmission path, the second transmission path, to the decoupling unit 20 of the base unit 4. The data stream is decoupled again (e.g., the data stream is separated into the second control data ZSD and the image data BD). The second control data ZSD is subsequently forwarded to the control unit 8, and the image data BD is forwarded to the image processing unit 10.

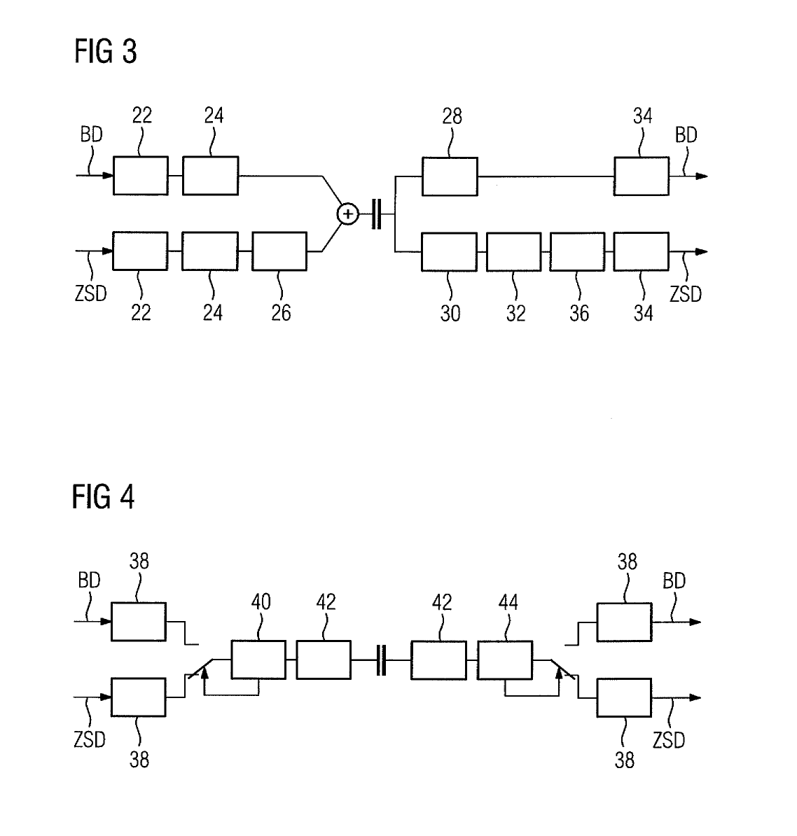

An advantageous implementation of the second transmission path is shown in FIG. 3 in the form of a block diagram. With this transmission path, the transmission of the data takes place in a contactless manner by a capacitive coupling, and the data transfer takes place with a frequency offset. This provides that the second control data ZSD and the image data BD are transmitted in parallel. Each of the two data streams has its own frequency band. Depending on the way in which the data has reached the second transmission path, the two data streams are first prepared according to a principle that is known with the aid of a clock recovery unit 22 (e.g., clock/data recovery (CDR)) and a low-pass filter 24. Subsequently, the second control data ZSD is upmodulated to a carrier frequency by a modulator unit 26. This modulated signal is added to the signal of the image data BD, and the composite signal is transmitted via the capacitive coupling on the fixed base unit 4. The transmission of the base data BD takes place in the baseband. The separation of the data into the image data BD and modulated signal is performed at the fixed base unit 4 side by a low-pass filter 28 and a bandpass filter 30. The modulated signal is demodulated with the aid of a demodulator unit 32, and the two data streams are processed by clock recovery units 34 and low-pass filters 36.

FIG. 4 shows an alternative embodiment of the second transmission path. The image data BD and the second control data ZSD each first go to a buffer memory 38 that operates in accordance with the “first in—first out” (FIFO) or “first-come first-served” (FCFS) principle. The two data streams are read out in a periodic sequence with the aid of a multiplexer, then transmitted via a capacitive coupling on the fixed base unit 4 and fed with the aid of a demultiplexer to two further buffer memories 38. The image data BD and the second control data ZSD are thus ultimately available as separate data streams in the fixed base unit 4.

The multiplexer includes a data packet generator 40 that takes a given data volume from the buffer memory 38 and sends the data volume to a serializer/deserializer unit 42 (SerDes) that converts the data packet data into a serial data stream. Depending on the size of the data packet (e.g., the volume of data), the transmission of this data stream requires a certain amount of time. Therefore, a corresponding time window is provided for the transmission. In one embodiment, the data packet generator 40 always generates data packets of the same size, so that each time window has the same temporal extension. The typically higher data rate for the image data BD is taken into account by the fact that, for example, only every third time window is used for the transmission of a data packet with second control data ZSD. The other time windows are reserved for data packets with image data BD. The demultiplexer in the fixed base unit 2 includes a further serializer/deserializer unit 42 for the restoration of the originally parallel data stream and a receive control 44 that assigns the data packets to the corresponding buffer memories 38.

The invention is not restricted to the exemplary embodiments described above. Instead, the person skilled in the art may also derive other variants of the invention without departing from the subject matter of the invention. For example, all individual features described in conjunction with the exemplary embodiments may also be combined in other ways with each other without departing from the subject matter of the invention.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical system comprising:
a fixed base unit comprising a control unit, and
a mobile assembly moveable relative to the fixed base unit,
wherein the fixed base unit and the mobile assembly are configured to exchange data, and
wherein transmission of first control data from the control unit to the mobile assembly takes place via a first transmission path, and transmission of second control data to the control unit and transmission of measured data from the mobile assembly takes place via a second transmission path.

2. The medical system as claimed in claim 1, wherein the medical system is a diagnostic system or a therapeutic system.

3. The medical system as claimed in claim 1, wherein the mobile assembly is mounted rotatably on the fixed base unit.

4. The medical system as claimed in claim 1, wherein the measured data comprises image data of an imaging system.

5. The medical system as claimed in claim 1, wherein the first transmission path and the second transmission path are provided as unidirectional transmission paths.

6. The medical system as claimed in claim 1, wherein the transmission of the first control data or the transmission of the second control data and the measured data takes place via the first transmission path or the second transmission path, respectively, in a contactless manner.

7. The medical system as claimed in claim 6, wherein the transmission of the first control data via the first transmission path, the transmission of the second control data and the measured data via the second transmission path, or the transmission of the first control data via the first transmission path and the transmission of the second control data and the measured data via the second transmission path is by capacitive coupling.

8. The medical system as claimed in claim 1, wherein the second control data and the measured data are transmitted with a frequency offset via the second transmission path.

9. The medical system as claimed in claim 8, wherein the second control data is modulated up to a carrier frequency for transmission via the second transmission path.

10. The medical system as claimed in claim 1, wherein the second control data and the measured data are transmitted with a time-offset via the second transmission path.

11. The medical system as claimed in claim 10, wherein the second control data and the measured data are each divided into data packets,
wherein for the time-offset transmission, two time-window types with different extension are arranged consecutively in a periodic sequence, a first time-window type of the two time-window types being provided for the second control data and a second time-window type of the two time-window types being provided for the measured data, and
wherein one of the data packets is transmitted in each time window.

12. The medical system as claimed in claim 9, wherein the second control data and the measured data are each divided into data packets,
wherein time windows of same extension are arranged consecutively for the time-offset transmission,
wherein one of the data packets is transmitted in each of the time windows, and
wherein the data packets of the second control data are transmitted in a periodic sequence.

13. The medical system as claimed in claim 1, wherein the second control data and the measured data are each buffered in a buffer memory that is accessed by a data packet generator such that, in a periodic sequence, either data from the buffer memory for the second control data or data from the buffer memory for the measured data are combined to form a data packet that is transmitted in a time window with a given extension.

14. The medical system as claimed in claim 1, wherein the medical system is embodied as a computed tomography system.

15. The medical system as claimed in claim 4, wherein the first transmission path and the second transmission path are provided as unidirectional transmission paths.

16. The medical system as claimed in claim 4, wherein the transmission of the first control data or the transmission of the second control data and the measured data takes place via the first transmission path or the second transmission path, respectively, in a contactless manner.

17. The medical system as claimed in claim 5, wherein the transmission of the first control data or the transmission of the second control data and the measured data takes place via the first transmission path or the second transmission path, respectively, in a contactless manner.

18. The medical system as claimed in claim 4, wherein the second control data and the measured data are transmitted with a frequency offset via the second transmission path.

19. The medical system as claimed in claim 7, wherein the second control data and the measured data are transmitted with a frequency offset via the second transmission path.

20. The medical system as claimed in claim 4, wherein the second control data and the measured data are transmitted with a time-offset via the second transmission path.
